(19) United States
(12) Patent Application Publication
(10) Pub. No.: US 2002/0142362 A1
(43) Pub. Date: Oct. 3, 2002

(54) Colorimetric assay system using thiopeptolide substrate for detection of membrane-type matrix metalloproteinase

(76) Inventors: Rosemarie Beth Flick, Copiague, NY (US); Bruce Alan Pfeffer, Valley Cottage, NY (US); Naveed Bin Kamal Shams, Duluth, GA (US); Stephen Paul Bartels, Wyckoff, NJ (US)

Correspondence Address: Rita D Vacca, Bausch & Lomb Inc, One Bausch & Lomb Place, Rochester, NY, 14604-2701, US

(21) Appl. No.: 09/576,501

(22) Filed: May 23, 2000

(51) Int. Cl. C12Q 1/37

(52) U.S. Cl. 435/23; 435/125

(57) ABSTRACT

A calorimetric assay system to measure the expression or activity of membrane-type matrix metalloproteinases using a thiopeptolide substrate. The subject colorimetric assay system is useful in screening pharmacological agents that may have therapeutic potential in the treatment of glaucoma and the promotion of wound healing. The subject colorimetric assay system is also useful in the discovery of new therapies for cancer and conditions involving neovascularization based on modulation of extracellular matrix.

### FIELD OF THE INVENTION

The present invention relates to a colorimetric assay system and a method for making and using the same. More particularly, the present invention relates to a colorimetric assay system that uses a thiopeptolide substrate for detecting cell-associated gelatinases in cells and tissues useful in the screening of pharmacological agents having therapeutic potential in the treatment of glaucoma and the promotion of wound healing. The subject calorimetric assay system may also be used in the discovery of new therapies for cancer and conditions involving neovascularization, based on modulation of extracellular matrix.

### BACKGROUND OF THE INVENTION

The extracellular matrix (ECM) is composed primarily of collagens, elastin, proteoglycans, and other glycoproteins. By virtue of the physico-chemical properties of these macromolecules, the ECM provides structural support and flexibility to tissues and organs (as in cartilage), mediates cell anchoring and migration (as in basement membranes of epithelial and endothelial cells), sequesters growth factors and transport proteins (as in the interphotoreceptor matrix of the retina), and creates specialized biochemical domains (as in the vitreous of the eye).

To maintain homeostasis from the cellular level upward, the synthesis, degradation, remodeling and assembly of ECM components must be well regulated. A crucial element in the breakdown of ECM molecules is the regulation of matrix metalloproteinases (MMPs). MMPs are members of a large gene family that includes collagenase, stromelysin, matrilysin, and gelatinases. Gelatinases include for example Gelatinase A (MMP-2; 72 kD gelatinase; type IV collagenase; E.C. 3.4.24.24), Gelatinase B (MMP-9; 92 kD gelatinase) and Membrane-Type MMPs (MT-MMPs). MT-MMPs are integral membrane proteins. To date there are five known MT-MMPs encoded in five separate genes as described by Takino et al., Gene 155, 293 (1995); Will and Hinzmann, Eur. J. Biochem. 231, 602-608 (1995); Takino et al., J. Biol. Chem. 270, 23013-23020 (1995); Puente et al., Cancer Res. 56, 944-949 (1996) and Pei, J. Biol. Chem. 274, 8925-8932 (1999).

As part of the regulatory mechanisms governing MMP activity, and hence ECM turnover, Gelatinase A is secreted in latent form as a proenzyme that in vivo requires proteolytic cleavage of a segment of amino acids for initiation of activation. Initial cleavage yields an intermediate form (64 kD), which undergoes further autocatalytic proteolysis to more active smaller molecular weight forms of 58 kD and 45 kD, the latter continuing to spontaneously degrade to inactive fragments. The in vitro activation of proGelatinase A may be effected by incubation with organomercurials such as for example p-aminophenyl mercuric acetate (APMA), but not by trypsin, as is the case with proGelatinase B. In vivo, the initial cleavage of proGelatinase A is carried out when the molecule encounters MT-MMP expressed on cell surfaces as described by Sato et al., Nature 370, 61-65, (1994). Through this mechanism, it has been verified that most of the currently identified MT-MMPs ultimately participate in the regulation of ECM degradation by its specific activation of proGelatinase A.

Perturbation of expression of MMPs may contribute to the etiology of many diseases. The overexpression of MT-MMP is believed to be an underlying factor in the pathogenesis of arthritis and cancer, to name two prominent examples. In fact, a distinct correlation may be made between levels of MT-MMP expression and the metastatic potential of malignant tumor cells as described by Sato and Seiki, J. Biochem. 119, 209-215 (1996). The migration of metastatic cells from a tumor is believed to depend on the degradation of ECM, such as basement membrane, and this is accomplished by MT-MMP-activated Gelatinase A. Also, a prevailing theory of the mechanisms underlying primary open-angle glaucoma cites an accumulation of ECM, at the level of the trabecular meshwork (TM), which impedes aqueous outflow, as a result of lowered MMP activity. Because Gelatinase A is important in the regulation of ECM by TM cells, it follows that a decrease in MT-MMP activity and/or expression might upset the balance between synthesis and degradation of ECM in the direction of abnormal accumulation of ECM components. By the same token, an accumulation of ECM not directly resulting from decreased endogenous MT-MMP expression might be alleviated by up-regulation of this molecule by TM cells.

Assays have been developed in the past to test for the presence of MMPs. A spectrophotometric assay for type-L collagenase (MMP-1) utilizing synthetic thiopeptolide substrate acetyl-prolyl-leucyl-glycyl-[2-mercapto-4-methyl-pentanoyl]-leucyl-glycyl-ethyl ester was developed by Weingarten and Feder as reported in Anal. Biochem. 147, 437-440 (1985). Interestingly, Weingarten and Feder showed that the cleaved thiopeptolide reacted with thiol reagents to give a product that not only absorbed at 410 nm, but also in the ultraviolet range at 324 nm. Weingarten and Feder also showed that the relative rates of hydrolysis of the thiopeptolide by various non-MMP classes of proteolytic enzymes were insignificant. Studies have also suggested that the thiopeptolide, used in the Weingarten and Feder assay, could be used as a substrate for additional MMPs, namely, macrophage elastase, MMP-2 (Gelatinase A), stromelysin, and MMP-9 (Gelatinase B) as described by Shipley et al., J. Biol. Chem. 271, 4335-4341 (1996); Xia et al., Biochem. Biophys. Acta 1293, 259-266 (1996); Ye et al., Biochemistry 31, 11231-11235 (1992); and Ye et al., Biochemistry 34, 4702-4708 (1995).

Another assay methodology developed for MMPs, uses the induction of mRNA for MT-MMP from fibroblasts or HT-1080 fibrosarcoma cells stimulated with Concanavalin A (Con A) as described by Lohi et al., Eur. J. Biochem. 239, 239-247 (1996). MT-MMP was also detected via Western blotting using polyclonal antibodies to peptide fragments of the protein. It was found that there was an apparent constitutive expression of a 60 kD form of MT-MMP, with an additional 43 kD form expressed as a result of exposure to Con A.

Zymography has also been used to display activation of proGelatinase A in conditioned media from Con A-treated cells as described by Ward et al., Biochem. Biophys. Acta 1079, 242-246 (1991), and is considered evidence of the expression of MT-MMP. It is important to note that some of the findings described above appeared in print before MT-MMP was characterized. Nevertheless, in retrospect, it is clear that the observed activation of proGelatinase A is due to the action of MT-MMP. Such activation of proGelatinase A was detected using autoradiography of protein blots as described by Brown et al., Kidney Intl. 43,163-170 (1993).

Fluorigenic peptide substrates have been developed for many of the MMPs as described by Bickett et al., Anal. Biochem. 212, 58-64 (1993). These fluorigenic peptide substrates have been utilized for detection or measuring activity of soluble forms, usually a recombinantly expressed catalytic fragment, of MT-MMP as described by Will et al., J. Biol. Chem. 271, 17119-17123 (1996) and Lichte et al., FEBS Left. 397, 277-282 (1996). It is possible that a cell-based fluorimetric assay making use of an attached monolayer of cells in a tissue-culture plastic multiwell plate might be subject to quenching and other forms of interference from cells and/or plate material.

Although assays for MMPs have been previously developed as noted above, the known assays have numerous limitations. Assays that depend on the detection of MMP mRNA or protein, or on the detection of MMP activated Gelatinase A, are labor-, material-, and equipment-intensive as well as time consuming. Assays that are not directly cell-based may not be able to detect changes in cell-based activity with sensitivity or efficiency. Also, known assays are relatively non-dynamic in that the same are not capable of being assessed over time and are not conducive to statistical computation of the linear increase in OD. Additionally, known assays that detect MMPs immunologically, detect total MMPs, which may include inactive and latent molecules that are still being processed within the cell.

Because of the noted shortcomings of current assays for MMPs, there is a particular need to be able to easily and accurately assess the activity or quantitative level of MT-MMP expression in the TM and like cells and tissues. Such an assay could not only assist in directly determining the expression of MT-MMP-like activity on tumors, but could also be used for the purposes of screening substances that, in model systems, might have potential as pharmaceutical agents to either increase or decrease expression and/or activity of MT-MMP. Such agents could be useful in the development of therapies for many diseases, cancer and arthritis being but two prominent examples.

### SUMMARY OF THE INVENTION

A colorimetric assay system for the quantitative detection of membrane type - matrix metalloproteinases (MT-MMPs) in cells and tissues made in accordance with the present invention uses a chromogenic substrate, namely, the synthetic thiopeptolide substrate acetyl-prolyl-leucyl-glycyl-[2-mercapto-4-methyl-pentanoyl]-leucyl-glycyl-ethyl ester. MT-MMPs as used herein means any of the MT-MMP molecules known or to be discovered, as well as any similar membrane-associated metalloproteinase activity that can be measured using the subject chromogenic substrate.

The calorimetric assay system of the present invention utilizes a solution of thiopeptolide substrate and a thiol reagent, and an assay buffer in which the biological sample has been equilibrated. The assay reaction begins when the thiopeptolide substrate/thiol reagent solution is thoroughly mixed with the assay buffer having the biological sample therein. The reaction is then allowed to progress to a suitable endpoint beyond which no further color reaction proceeds. At this point, the amount of MT-MMP present on the biological sample and the effects of experimental conditions on the expression of MT-MMP may be determined as described in greater detail below.

Accordingly, it is an object of the present invention to provide an assay specific for MT-MMP.

Another object of the present invention is to provide an assay capable of quantitatively measuring membrane-associated metalloproteinase activity.

Another object of the present invention is to provide an assay capable of quantitatively measuring membrane-associated metalloproteinase activity in cells and/or tissues.

Another object of the present invention is to provide an assay for MT-MMP useful in screening potential pharmaceutical agents that increase or decrease the activity of MT-MMP.

Another object of the present invention is to provide an assay that is reliable and easy to use.

Still another object of the present invention is to provide an assay that is colorimetric for ease of use.

These and other objectives and advantages of the present invention, some of which are specifically described and others that are not, will become apparent from the detailed description and claims that follow.

### DETAILED DESCRIPTION OF THE INVENTION

A calorimetric assay system for detecting cell-associated metalloproteinases in cells and tissues in accordance with the present invention, uses a thiopeptolide substrate, namely, the synthetic thiopeptolide substrate acetyl-prolyl-leucyl-glycyl-[2-mercapto-4-methyl-pentanoyl]-leucyl-glycyl-ethyl ester, and a thiol reagent solution, and an assay buffer as described in detail herein.

The assay of the present invention may be made by first preparing the selected biological sample and assay buffer. To prepare the biological sample, the biological sample such as but not limited to cultured cells were placed in microtiter plates and incubated at approximately 37° C. for up to approximately 96 hours with serum-free, defined medium. A portion of the serum-free, defined medium contained test compounds, while another portion was maintained free of test compounds to serve as controls. In some cases, the biological sample was then processed as a permeabilized “ghost” as described in Example 1 below. The biological sample was then rinsed several times with cold isotonic balanced saline or Tris(hydroxymethyl)aminomethane (TRIS) buffer at pH 7.2 - 7.5. The saline or buffer was then replaced by filter-sterilized, isotonic assay buffer (295 mOsm) consisting of approximately 50 mmol/L N-[2-hydroxyethyl]piperazine-N′-[2-ethane sulfonic acid] (HEPES), approximately 106 mmol/L sodium chloride, approximately 3.5 mmol/L potassium chloride, approximately 5 mmol/L calcium chloride, and approximately 0.02% (v/v) polyoxyethylene 23 lauryl ether (Brij 35) or a similar detergent, final pH approximately 7.5, at approximately room temperature. The Brij 35 was added fresh, and the final mixture was protected from excessive light exposure. The volume of assay buffer for each biological sample was approximately one-half the final assay mixture volume, the other half being made up of the thiopeptolide substrate/thiol reagent solution. For example, when 96-well microtiter plates were employed, the assay mixture volume was approximately 200 microliters.

Next the thiopeptolide substrate/thiol reagent solution was prepared. Preferably, the synthetic thiopeptolide substrate acetyl-prolyl-leucyl-glycyl-[2-mercapto-4-methyl-pentanoyl]-leucyl-glycyl-ethyl ester (Bachem Bioscience, Inc., King of Prussia, Pa.) is used in the solution due to its ready availability. The synthetic thiopeptolide was stored as a 40X, 20 mmol/L stock in DMSO, sealed from light and air, and stored with desiccant at approximately −70° C.. The thiol reagent, 4,4-dithiodipyridine,5,5′-dithio bis(2-nitro-benzoic acid) (DTNB) or the like, but preferably DTNB, was prepared as a 100X, 50 mmol/L stock in DMSO, and stored protected from light with desiccant at approximately room temperature. The combined thiopeptolide and DTNB were both diluted to appropriately 1 mmol/L in assay buffer without Brij 35 to yield a solution that was 2X for both chemicals after thorough mixing. The assay reaction was started when this 2X solution was added in a 1:1 ratio, with thorough mixing, to the assay buffer in which the cells had been equilibrated. The final concentration of both thiopeptolide and DTNB were 0.5 mmol/L.

The assay reaction was then allowed to progress to a suitable endpoint beyond which no further color reaction proceeded, possibly due to product inhibition. This endpoint is usually reached after approximately two hours. During the two hours the samples were gently agitated in an approximately 37° C. humidified incubator. Once the endpoint was reached the microtiter plates were placed into a plate reader and optical density (OD) was measured at 410 nm. Blank values, the OD readings for samples without cells, used to assess non-enzymatic, non-specific hydrolysis of substrate, were then subtracted from all sample OD measurements. The OD measurements taken at 410 nm may be used as a measure of the amount of MT-MMP present on the samples. The effects of experimental conditions on the expression of MT-MMP may then be compared with that of the control samples.

The assay of the present invention may also be carried out by following the reaction kinetics in the spectrophotometric plate reader. In this case, v, defined as the rate of appearance of the product of the reaction catalyzed by MT-MMP, is proportional to the increase in OD as a function of time. The slope of the linear portion of this plot may be used as a measure of MT-MMP activity in the samples.

The subject assay is described in still greater detail in the examples provided below which in no way are intended to limit the scope of the present invention.

### EXAMPLE 1-Thiopeptolide Assay on Cultured Trabecular Meshwork Cells Incubated with Concanavalin A to Measure the Effects of Inhibitors of Transcription and Translation:

Zymographic profiles of conditioned medium from control TM cells in culture revealed negligible levels of active Gelatinase A, suggesting low constitutive MT-MMP activity. Incubations with lectin Concanavalin A (Con A) induced robust activation of proGelatinase A in cultured TM cells attributed to the enhanced expression of MT1-MMP, as described above. Details of the signal transduction process initiated by Con A culminating in proGelatinase A activation by MT-MMP remained to be elucidated. By incubating both control and Con A-treated, non-human primate TM cell cultures with either actinomycin D—an inhibitor of messenger RNA transcription from DNA—or cycloheximide--an inhibitor of translation of protein from messenger RNA—the dependence of the Con A-induced expression of MT-MMP on these two synthetic processes was determined, by means of the herein described cell-based thiopeptolide assay.

Rhesus monkey TM cells in third passage were plated in 96-well Primaria™ (Collaborative Biomedical, Bedford, Mass.) microtiter plates and maintained for up to two weeks at confluence in growth medium containing 1% calf serum, until commencement of the experiment. Cells were conditioned for three days with the above growth medium without serum, and then rinsed several times with a more simplified, totally defined medium, Concanavalin A Conditioning Medium (CACM), that permits optimal responses to Con A. Duplicate wells then underwent one of seven protocols as set forth in Table 1 below. At the end of these incubations, the media were removed from the wells, the cells washed twice with CACM, and then permeabilized with digitonin. Permeabilization involved preincubation for 20 to 30 minutes with three changes of a cold solution of potassium acetate (110 mmol/L), magnesium acetate (2 mmol/L) in HEPES buffer (20 mmol/L, pH 7.2). The cells were then treated with 40 ug/ml digitonin in the same buffered solution for five minutes on ice, after which the digitonin was rinsed out with three changes of a wash buffer consisting of potassium acetate (90 mmol/L) in HEPES buffer (50 mmol/L, pH 7.2). The rinse out period was 20 to 30 minutes, followed by a short equilibration with assay buffer, culminating in the thiopeptolide assay, performed on the cells as described above.

TABLE I

| Sample | | |
|---|---|---|
| No. | Preincubation, 1 hour | Next 24 hours |
| 1. | CACM | Control CACM |
| 2. | CACM | Con A (10 ug/ml) in CACM |
| 3. | Actinomycin D, 500 ng/ml | Act D |
| | in CACM (Act D) | |
| 4. | Cycloheximide (20 ug/ml) | CHX |
| | in CACM (CHX) | |
| 5. | Act D | Con A plus Act D |
| 6. | CHX | Con A plus CHX |
| 7. | CACM | Con A 6 for hours, then |
| | | Con A plus Act D for 18 hours |

Results from the seven protocols described in Table 1 above are illustrated in Chart 1 below. Values for duplicate wells for each sample number are represented separately by total bar heights and by dark-shaded bar heights in Chart 1. Cells incubated with control CACM alone generated a final OD reading in the thiopeptolide assay commensurate with low levels of expression of MT-MMP and/or non-specific proteolytic hydrolysis of thiopeptolide substrate. In contrast, Con A incubation generated MT-MMP expression of more than 200% over control levels. Incubation of either control or Con A-treated cells with CHX gave results not significantly different from control CACM alone, as was also the case with control cells treated with Act D. When cells were incubated with Con A plus Act D, or incubated with Con A for 6 hrs before Act D co-administration, the MT-MMP activity as determined by the thiopeptolide assay was slightly, but discernibly, higher than control. There was excellent agreement between the duplicate values for each experimental condition, attesting to the reliability of the assay.

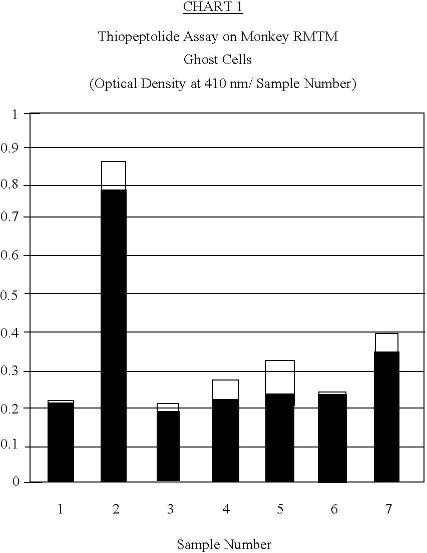

The thiopeptolide assay was clearly sensitive and interpretable over the broad range between the relatively low level of expression of MT-MMP in control cells and the greatly increased expression generated by 24 hours of treatment with 10 ug/ml Con A. The results also showed that protein synthesis was necessary for Con A to exert its stimulation of MT-MMP expression. Furthermore, the assay could be used to detect the slight penetrance of expression of MT-MMP when cells were incubated concurrently with Con A plus Act D, or when six hours of Con A treatment was followed by incubation with Act D in the continued presence of Con A. One possible explanation of these last findings is that exposure of cells to Con A and Act D resulted in the translation of a small pool of pre-existing, but short-lived mRNA transcripts, and that a short incubation with Con A alone permitted the initial generation of a relatively small number of new MT-MMP message transcripts before Act D subsequently shut down mRNA synthesis.

### EXAMPLE 2-Thiopeptolide Assay on Excised Trabecular Meshwork from Human Cadaver Eyes:

It is important to verify that assay results from a pure preparation of cultured TM cells can be extended to intact trabecular meshwork. Accordingly, an explanted human anterior segment organ culture system containing native TM was utilized with the subject thiopeptolide assay to obtain results of incubation with Con A with respect to the expression of MT-MMP or a corresponding activity. Consequently, a comparison between the system's response and that of the pure TM cells established in subculture could be made. Although some changes may take place during the three days after explanting the samples, it was expected that the results would provide a measure of how closely the properties of TM cells in long-term culture correspond to those of native TM tissue.

A pair of human donor eyes from a 52 year old donor were enucleated approximately two and a half-hours post mortem. The anterior segments, with the lens removed, were isolated and shipped overnight in Optisol™ (Bausch & Lomb Surgical, Inc., Claremont, Calif.) to the laboratory, where both segments were dissected into quadrants. The quadrants were placed in defined culture medium and incubated for four hours in a humidified incubator at 370° C.. Each of the eight quadrants was then transferred to an individual well of a 12-well cluster containing defined culture medium either without added Con A (control) or with 10, 20, 30, 40, 60, 60, or 100 ug/ml Con A. The incubation volume was 0.5 ml. Twenty-four hours later, the media was replaced with equivalent experimental media, and the segments underwent a further eighteen-hour incubation. Then strips of TM were dissected from each quadrant. The dissections were performed with balanced saline containing 0.3 mg/ml bovine serum albumin. Within two hours of the end of the experimental incubation the strips were placed, using sterile plastic transfer pipettes, into 24-well clusters, given two short rinses in CACM, one rinse in thiopeptolide assay buffer, and then placed into the thiopeptolide assay mixture described above, with the reaction volume being 300 uL. Since the plate reader in use only accommodated 96-well microtiter plates, after a three hour reaction, with gentle agitation in a humidified incubator, approximately 200 uL from each sample were transferred from the 24-well clusters to the 96-well format for OD readings as described above.

Results from Example 2 are shown in Chart 2 below. In this dose-range study of the effects of Con A on expression of MT-MMP in intact TM, it can be seen that a threshold was reached at a Con A concentration of 30 ug/ml, below which the results were indistinguishable from simply incubating anterior segment quadrants in control medium. The response from 30 to 100 ug/ml Con A was not linear, with 30 - 80 ug/ml Con A yielding fairly close values, approximately 100% over control. The highest OD readings, 200% over control, were seen with 100 ug/ml Con A.

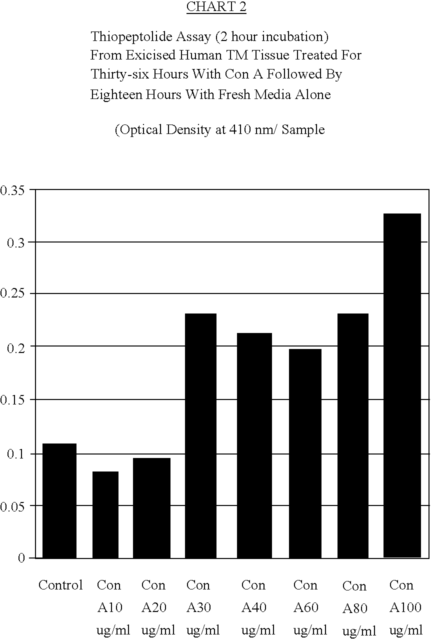

Although there was only one sample per data point representing a range of eight different levels of Con A treatment, including a control without Con A, the results show that, in general, increasing Con A concentrations led to corresponding increases in OD from the thiopeptolide reaction. This is interpreted as a direct correlation with the amount of MT-MMP, or with our present knowledge, MT1-MMP expressed in the TM of the organ culture preparations. The OD values at the end-point of the incubation period were used for quantifying MT-MMP. This is considered to be representative of the comparative rates of reaction for each of the samples.

Although the TM samples certainly contained live cells, arguably capable of producing Gelatinase A as well as MT-MMP, any Gelatinase A produced would have utilized the thiopeptolide substrate. However, the color development after two hours was almost entirely due to MT-MMP expression on the surface of the cells. One piece of evidence to support that almost all color development was due to MT-MMP expression is that one- or two-day conditioned medium from equivalently treated TM strips was ineffective in the thiopeptolide reaction. Additionally, zymography displayed only very faint bands corresponding to either proGelatinase A or activated Gelatinase A. This suggests that during the two-hour incubation in assay medium, soluble or even membrane-associated MMPs, besides MT-MMP, could not have accumulated in amounts significant enough to contribute to the proteolysis of the thiopeptolide substrate.

Human fibrosarcoma cell line HT-1080 can be stimulated by in vitro incubation with phorbol ester (phorbol 12-myristate 13-acetate; PMA), and with Con A, to activate Gelatinase A from the proenzyme form (as assessed by zymography), and to increase expression of MT1-MMP (as assessed by Western immunoblot), as described by Lohi et al., Eur. J. Biochem. 239: 239-247; Lehti et al., Biochem. J. 334: 345-353 (1998); and Stanton et al., J. Cell Science 111: 2789-2798 (1998). The activation of proGelatinase A was shown in the noted Lohi and Stanton work to be inhibitable by tissue inhibitor of [matrix] metalloproteinases-2 (TIMP-2), and by a synthetic hydroxamate inhibitor of MMP's, but not by metalloproteinases-1 (TIMP-1). As additional evidence that the subject assay detects MT1-MMP in human and non-human primate TM cells, the zymographic results of the Lohi and Stanton work were reproduced using the subject assay, using the same methodology as described herein for TM cells, with HT-1080 cells and HT-1080 cells. Increased activity in the subject thiopeptolide assay following treatment of HT-1 080 cells with PMA or Con A was demonstrated.

Cultured monkey TM cells were also found to express messenger RNA for MT1 -MMP by standard molecular genetic techniques using specific nucleic acid primers for human MT1-MMP, verified by subsequent sequencing of the major transcript that was expressed at the expected and appropriate base pair molecular size for MT1-MMP. When the cultured monkey TM cells were treated with agents previously demonstrated to increase the activation of proGelatinase A, as shown by zymography, an increase in expression of MT1-MMP protein in cell extracts was demonstrated by Western blotting. In the latter procedure an antibody specific for MT1-MMP was employed to label only the MT1-MMP protein after it had electrophoretically migrated in a polyacrylamide gel to a position appropriate for its molecular size. Again, results were obtained similar to those depicted in Lehti, et al., (1998) for HT-1080 cells. Furthermore, the zymographic and immunoblotting results correlated precisely with the subject thiopeptolide assay results from the TM cells used in parallel experiments. In these parallel experiments, the treatment of the biological sample with agents that increase activation of proGelatinase A and increase MT1 -MMP protein brought about a nearly four-fold increase in the thiopeptolide assay results.

In using the subject thiopeptolide assay on cultured TM cells, it was found that activity increases in the subject thiopeptolide assay were inhibitable by TIMP-2 and a hydroxamate MMP inhibitor, but not by TIMP-1. Evidence has been presented by Mazzieri, et al., EMBO Journal 16: 2319-2332 (1997) that the urokinase-plasmin system may be in part responsible for activation of proGelatinase A in some cell systems. It was found in studying the subject thiopeptolide assay that a lesser fraction of the subject thiopeptolide assay activity could be inhibited by serine protease inhibitors, which are ineffective with MMP's. However, when these latter inhibitors were employed with TM cells stimulated by Con A, the percentage increase in thiopeptolide assay activity, compared to control cultures, was in fact augmented due to the Con A treatment. This shows that the subject thiopeptolide assay experiences only minimal if any interference from endogenous inhibitors as is desired.

The subject thiopeptolide assay as described above may be prepared in the form of a kit for ease and efficiency in measuring the expression or activity of MT-MMPs on a biological sample. The preferred assay kit has a removably sealable first container or vial, such as but not limited to a bottle with a cap having male and female threaded means of sealing engagement, although other configurations known to those skilled in the art could be used. The container and cap may be made of the same or different suitable materials such as but not limited to glass, plastic or metal. The container or vial is at least partially filled with an effective volume of substrate capable of being cleaved by MT-MMPs to result in a colored product and an effective volume of corresponding reagent. After the container or vial is filled with the substrate and reagent solution, the same is sealed. Also included in the preferred assay kit is a removably sealable second container or vial at least partially filled with a buffer solution prior to sealing. The configuration and/or materials of the second container and sealing means may be the same or different from those of the first container. Preferably the second container and sealing means is the same or similar to that of the first container and sealing means for ease of use. Preferably the second container or vial has a capacity large enough to simultaneously hold the contents of the first container, the contents of the second container and a prepared biological sample. After both the containers or vials have been sealed, both are packaged in a common container such as a tray and/or a box.

Such an assay kit of the present invention is prepared by obtaining a removably sealable first container or vial with sealing means. The first container in its unsealed state is at least partially filled with a solution of a substrate capable of being cleaved by membrane-type matrix metalloproteinases to result in a colored product and a corresponding reagent and then sealed. A removably sealable second container or vial with sealing means is obtained. The second container, in its unsealed state, is at least partially filled with a buffer solution and is then sealed. The assay kit is then completed by packaging the first sealed vial and the second sealed vial in a common package.

The assay kit described above may be used by removing the cap or seal from the first container or vial filled with the substrate and corresponding reagent solution. Then the cap or seal from the second container or vial filled with the buffer solution is removed. A prepared biological sample is placed in the second vial filled with the buffer solution and the contents of the first vial are added to the contents of the second vial. The expression or activity of MT-MMPs present on said biological sample may then be measured as described above.

The subject MT-MMP assay as described in detail herein is less labor-, material-and equipment-intensive than known assays that are dependent on the detection of MMP mRNA or protein, or on the detection of MMP activated Gelatinase A. The subject assay is likewise relatively sensitive and efficient in detecting changes in cell-based MT-MMP activity. Due to the kinetics of the MT-MMP-catalyzed proteolytic cleavage of the synthetic thiopeptolide substrate, acetyl-prolyl-leucyl-glycyl-[2-mercapto-4-methyl-pentanoyl]-leucyl-glycyl-ethyl ester, the same can be assessed over time making the subject assay more dynamic and more conducive to statistical computation of the linear increase in OD than currently known assays. Also, the subject assay only detects functional, active species of MT-MMP expressed on cell surfaces with minimal interference from endogenous inhibitors and does not detect inactive and latent molecules still being processed within a cell.

While there is shown and described herein certain specific embodiments of the present invention, it will be manifest to those skilled in the art that various modifications may be made without departing from the spirit and scope of the underlying inventive concept and that the same is not limited to the particular forms herein described except insofar as indicated by the scope of the appended claims.

We claim:

1. An assay for the measurement of expression or activity of membrane-type matrix metalloproteinases in biological samples comprising:
a solution of a substrate capable of being cleaved by membrane-type matrix metalloproteinases to result in a colored product and a corresponding reagent, and a buffer solution.

2. A method of preparing an assay for the measurement of expression or activity of membrane-type matrix metalloproteinases in biological samples comprising:
preparing a solution of a substrate capable of being cleaved by membrane-type matrix metalloproteinases to result in a colored product and a corresponding reagent, and preparing a buffer solution.

3. A method of using an assay for the measurement of expression or activity of membrane-type matrix metalloproteinases in biological samples comprising:
placing a biological sample in a buffer solution, adding a solution of a substrate capable of being cleaved by membrane-type matrix metalloproteinases to result in a colored product and a corresponding reagent to said buffer solution, waiting for completion of color change, and measuring color change to determine level of membrane-type matrix metalloproteinase activity or expression.

4. A kit for measuring expression or activity of membrane-type matrix metalloproteinases comprising:
a vial of a solution of a substrate capable of being cleaved by membrane-type matrix metalloproteinases to result in a colored product and a corresponding reagent, and a vial of a buffer solution.

5. A method of preparing a kit for measuring expression or activity of membrane-type matrix metalloproteinases comprising:
filling a removably sealable vial with a solution of a substrate capable of being cleaved by membrane-type matrix metalloproteinases to result in a colored product and a corresponding reagent prior to sealing the same, filling a removably sealable vial with a buffer solution prior to sealing the same, and packaging each vial in a common container.

6. A method of using a kit for measuring expression or activity of membrane-type matrix metalloproteinases comprising:
removing the seal from a removably sealable vial filled with a solution of a substrate capable of being cleaved by membrane-type matrix metalloproteinases to result in a colored product and a corresponding reagent, removing the seal from a removably sealable vial filled with a buffer solution, placing a prepared biological sample in said vial filled with buffer solution, adding the contents of said vial filled with substrate and corresponding reagent solution to said vial filled with a buffer solution, and measuring expression or activity of membrane-type matrix metalloproteinases present on said biological sample.

7. The assay of claim 1 wherein said substrate is thiopeptolide.

8. The assay of claim 1 wherein said substrate is acetyl-prolyl-leucyl-glycyl-[2-mercapto4-methyl-pentanoyl]-leucyl-glycyl-ethyl ester.

9. The assay of claim 1 wherein said reagent is thiol.

10. The assay of claim 1 wherein said reagent is 4,4-dithiodipyridine,5,5′-dithio bis(2-nitro-benzoic acid).

11. The assay of claim 1 wherein said buffer solution is a sterilized, isotonic assay buffer.

12. The assay of claim 1 wherein said buffer consists of N-[2-hydroxyethyl]piperazine-N′-[2-ethane sulfonic acid], sodium chloride, potassium chloride, calcium chloride, and detergent to achieve a final pH of approximately 7.5 at approximately room temperature.

13. The method of claim 2, 3, 5 or 6 wherein said substrate is thiopeptolide.

14. The method of claim 2, 3, 5 or 6 wherein said substrate is acetyl-prolyl-leucyl-glycyl-[2-mercapto-4-methyl-pentanoyl]-leucyl-glycyl-ethyl ester.

15. The method of claim 2, 3, 5 or 6 wherein said reagent is thiol.

16. The method of claim 2, 3, 5 or 6 wherein said reagent is 4,4-dithiodipyridine,5,5′-dithio bis(2-nitro-benzoic acid).

17. The method of claim 3, 4, 6 or 7 wherein said buffer solution is a sterilized, isotonic assay buffer.

18. The method of claim 3, 4, 6 or 7 wherein said buffer consists of N-[2-hydroxyethyl]piperazine-N′-[2-ethane sulfonic acid], sodium chloride, potassium chloride, calcium chloride, and detergent to achieve a final pH of approximately 7.5 at approximately room temperature.

19. The kit of claim 5 wherein said substrate is thiopeptolide.

20. The kit of claim 5 wherein said substrate is acetyl-prolyl-leucyl-glycyl-[2-mercapto-4-methyl-pentanoyl]-leucyl-glycyl-ethyl ester.

21. The kit of claim 5 wherein said reagent is thiol.

22. The kit of claim 5 wherein said reagent is 4,4-dithiodipyridine,5,5′-dithio bis(2-nitro-benzoic acid).

23. The kit of claim 5 wherein said buffer solution is a sterilized, isotonic assay buffer.

24. The kit of claim 5 wherein said buffer solution consists of N-[2-hydroxyethyl]piperazine-N′-[2-ethane sulfonic acid], sodium chloride, potassium chloride, calcium chloride, and detergent to achieve a final pH of approximately 7.5 at approximately room temperature.
